(19) United States
(12) Patent Application Publication
(10) Pub. No.: US 2011/0154543 A1
(43) Pub. Date: Jun. 23, 2011

(54) METHOD OF CULTIVATING GRAFTED CACTUS (GYMNOCALYCIUM MIHANOVICHII) CULTIVAR "YELLOW TOP" AND THE GRAFTED CACTUS CULTIVATED THEREBY

(75) Inventor: Chang-Hui JO, Goyang-si (KR)

(73) Assignee: GYEONGGI-DO, Suwon-si, (KR)

(21) Appl. No.: 12/965,189

(22) Filed: Dec. 10, 2010

Related U.S. Application Data
(60) Provisional application No. 61/282,112, filed on Dec. 17, 2009.

(51) Int. Cl. A01H 5/00; A01G 1/06

(52) U.S. Cl. 800/298; 47/6

(57) ABSTRACT

Provided herein is a method of cultivating a grafted cactus (Gymnocalycium mihanovichii) cultivar “Yellow Top” including the steps of: a) selecting a cross combination between “GG02451” and “GG205736”; b) crossing the combination, harvesting cactus fruits after a mature stage has been passed, disinfecting the cactus fruits, selecting and sterilizing seeds contained in the cactus fruits, and sowing the seeds; c) obtaining germinated seedlings; d) selecting only colored seedlings from the germinated seedlings; e) first-grafting the selected colored seedlings onto small virus-free Hylocereus trigonus in test tubes; and f) covering light-shielding film on the cultured seedlings to harden the seedlings and secondarily grafting only the seedlings onto Hylocereus trigonus.

### CROSS-REFERENCE TO RELATED APPLICATIONS

Pursuant to 35 U.S.C. §119(e), this application claims the benefit of U.S. Provisional Application Ser. No. 61/282,112, filed on Dec. 17, 2009 in the name of Chang-Hui Jo, which is entitled “A NOVEL CACTUS, GYMNOCALYCIUM MIHANOVICHII “YELLOW TOP.” The provisional application is hereby incorporated by reference as if it were fully set forth herein.

### BACKGROUND OF THE INVENTION

1. Field of the Invention

The present subject matter relates to a method of cultivating a grafted cactus cultivar and the grafted cactus produced thereby. In particular, the present subject matter relates to a method of cultivating a grafted cactus (Gymnocalycium mihanovichii) cultivar, named “Yellow Top” and the same grafted cactus cultivar cultivated by the method. The grafted cactus (Gymnocalycium mihanovichii) cultivar “Yellow Top” has a vivid yellow bulb and has good adaptability to growth environment to provide a high ornamental value.

2. Background of the Related Art

More than 200 genera of plants exist in the cactus family, Cactaceae. Cactuses are classified into succulent plants in view of their shape, but since the plants grouped in the Cactaceae have been grouped by their numerous kinds, cactuses are generally separated from the rest of succulent plants.

Most cactuses produce spines not from their leaves, but from their areoles, and they have spherical, flat-round, and cylindrical stems, which are very unique and are not found in general plants.

Since the cactuses conserve lots of water in their tissues for a long time, they can be adapted in states exposed to dry climate for a long time. Further, deep wrinkles are formed on the surfaces of most cactuses, and they serve as a radiator that adjusts the cactus's temperature to prevent the temperature from being exceedingly raised by radiant heat therearound.

The cactus has reticulate xylem looking beautiful, and thus, craft products are made with the reticulate xylem of the cactus.

The cactuses grow to a variety of shapes. For example, they grow to a ball-like shape, a rectangular shape, a pentagonal shape, large and small protrusions-branching shape, a long shape like a strap, and so on.

The areoles formed in the cactuses are standards in classifying the cactuses from the rest of succulent plants. That is, the spines of the cactuses are produced from the areoles, but the spines of the rest of succulent plants like “Crown of thorns” are the results of the growth of a portion of epidermis, while having no areoles formed thereon.

The spines of the cactuses have big differences in their color, length, thickness, and number in accordance with the species of the cactuses. Most cactuses have 1 or 2 large and strong central spines arising from the center of the areole and 5 to 20 small radial spines located around the central spines.

The flowers of the cactuses have various leaf colors and also distinct colors, so that they look more beautiful than those of other plants. The flowers of the cactuses generally have flowering period of more than one month, but some cactuses have flowering period continued during spring, summer and fall. However, the flowering period of a blossom of flower of the cactuses is shorter than that of other plants.

Grafted cactuses like Gymnocalycium mihanovichii, Chamaecereus silvestrii, and so on are cactus cultivars that are produced and sold by new cultivar cultivating technology in Korea, and they occupy about 70% of a world market of the grafted cactuses. Referring especially to the export percentages by colors of the grafted cactus, Gymnocalycium mihanovichii, red is 50%, yellow is 15%, orange is 15%, pink is 15%, and black and red is 5%. However, the color preferences of the grafted cactuses are different by countries, ages and sexes, and recently, the preferences for yellow, pink and orange colors are increased. Gymnocalycium mihanovichii used as a scion of the grafted cactus is a varitas separated from crossed seedlings and is not autotrophic because of nonexistence of chlorophyll. Thus, they are cultivated by taking a cormlet, i.e. dry set, and grafting the cormlet onto a green rootstock.

However, they are often infected with viruses due to the nutrition growth through the grafting, so that various problems have occurred in the colors and shapes of the grafted cactuses during their growth.

To solve these problems, there have been needs in the art for development of production technology for new cultivars having various bulb colors and shapes, and for improvement of various technologies to enhance ornamental values thereof.

The present inventor has conducted various researches and studies to develop a method for cultivating a new cactus cultivar providing a bulb of new color and shape, through stable reproduction. As a result, it was discovered a method for cultivating a new grafted cactus (Gymnocalycium mihanovichii) cultivar “Yellow Top” that has a vivid yellow bulb and has good adaptability to growth environment to provide a high ornamental value.

### SUMMARY OF THE INVENTION

Accordingly, the present subject matter has been made in view of the above-mentioned problems occurring in the prior art, and it is an object of the present subject matter to provide a new grafted cactus (Gymnocalycium mihanovichii) cultivar “Yellow Top” that has good adaptability to a growth environment to provide a high ornamental value. The grafted cactus has a yellow bulb and has no change in color by some environmental variation.

It is another object of the present subject matter to provide a method for cultivating a new grafted cactus (Gymnocalycium mihanovichii) cultivar “Yellow Top,” wherein progenies of the crosses of the cactuses contained in Gymnocalycium mihanovichii are crossed and cultivated through tissue culture technology using virus-free seedlings, and wherein the cultivating method comprise selecting cactus cultivars after the characteristics of the cactus cultivars have been investigated in a test greenhouse in accordance with their color uniformity, shape stability, differentiation, and the like. In the test, particularly the grafted cactus (Gymnocalycium mihanovichii) cultivar “Yellow Top” of the present subject matter has passed an adaptation test to cultivating sites of farms, such that the preferences of the cultivators and the adaptability to the environment are satisfied to increase the consumer's preference for the color uniformity, shape stability and ornamental value of the new cactus cultivar of the present subject matter.

To accomplish these objects, provided herein is a method of cultivating a grafted cactus (Gymnocalycium mihanovichii) cultivar “Yellow Top,” comprising: a) selecting a cross combination between Gymnocalycium mihanovichii “GG02451,” as a male sterile line (mother plant) having a yellow bulb and a green growing point, and Gymnocalycium mihanovichii “GG205736,” as a restorer line (father plant) having an orange bulb; b) crossing the combination, harvesting cactus fruits at a state of being not opened at the shells thereof after a mature stage has been passed, disinfecting the cactus fruits having the shells in a flame of ethanol, selecting and sterilizing seeds contained in the cactus fruits, and sowing the seeds in petri dishes in a clean-bench, each petri dish having medium 30 ml divided thereinto; c) culturing the sowed seeds to obtain germinated seedlings; d) selecting only colored seedlings from the germinated seedlings; e) first-grafting the selected colored seedlings onto small virus-free Hylocereus trigonus in test tubes, supportedly placing the Hylocereus trigonus onto which the seedlings are grafted in media divided into sterilized test tubes, and culturing the grafted seedlings in a culture room; and f) covering light-shielding film on the cultured seedlings to harden the seedlings, secondarily grafting only the seedlings onto Hylocereus trigonus grown to a length of 14 cm to 15 cm in an outside greenhouse, and cultivating the grafted seedlings in the outside greenhouse.

In an embodiment of the present subject matter, the secondarily grafting of the seedlings in (f) includes selecting the seedlings each having a diameter in a range of 0.7 cm to 1 cm.

In another embodiment, the obtaining of the seedlings in (c) is carried out in a constant temperature and humidity culture room of 26° C. under the conditions of 2,500 lux of illumination intensity and daylength of 16 hours.

### BRIEF DESCRIPTION OF THE DRAWINGS

The patent or application file contains at least one drawing executed in color. Copies of this patent or patent application publication with color drawings will be provided by the Office upon request and payment of the necessary fee.

The above and other objects, features and advantages of the present subject matter will be apparent from the following detailed description of the preferred embodiments in conjunction with the accompanying drawings, in which:

FIG. 1 is a photograph showing a front end portion of a grafted cactus (Gymnocalycium mihanovichii) cultivar “Yellow Top” cultivated by the method according to one aspect of the present subject matter; and

FIG. 2 is a photograph showing the side aspect of a grafted cactus (Gymnocalycium mihanovichii) cultivar “Yellow Top” according to the present subject matter grafted onto Hylocereus trigonus.

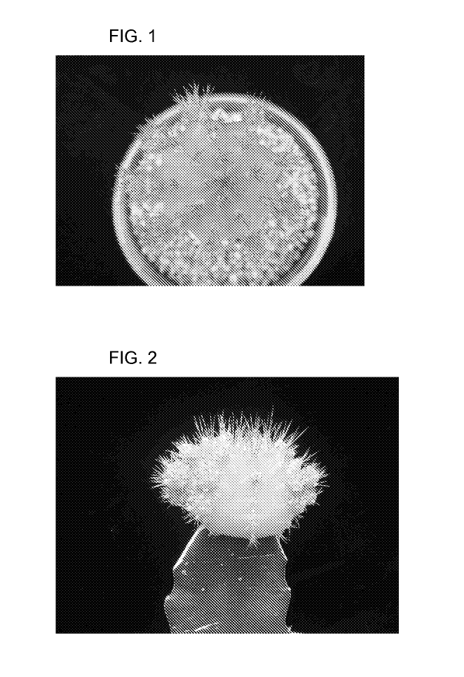

### DETAILED DESCRIPTION OF THE PREFERRED EMBODIMENT

The present subject matter is achieved by selecting and crossing cactuses having excellent characteristics from genetic resources collected so as to cultivate a new grafted cactus cultivar having a yellow bulb and no change in color by small environmental variation among the grafted cactuses Gymnocalycium mihanovichii.

First, Gymnocalycium mihanovichii “GG02451” used as a male sterile line (mother plant) has a yellow bulb and a green growing point, and Gymnocalycium mihanovichii “GG205736” used as a restorer line (father plant) has an orange bulb.

Cactuses maintain and reproduce their species through propagation by seed and vegetative propagation (asexual propagation). The propagation by seed is a process of obtaining seeds by crossing and sowing the seeds to produce new offspring, but since most cactuses are not possible to have self-fertilization, they have only cross-fertilization.

Through the self-fertilization, the genetic traits like the maternal side are expressed by the seeds, but since the cross-fertilization is achieved only by hybrid crossing, genetic segregation occurs such that the traits of the new cultivar are not expressed in next generation.

According to the present subject matter, the new cactus cultivar offshoots are grafted onto Hylocereus trigonus and have the vegetative propagation to pass the same genetic traits to the next generation. Especially, since the grafted cactus of the present subject matter is propagated through the vegetative propagation (grafting), the same characteristics are expressed again to the next generation of the new cactus cultivar.

According to the present subject matter, crossing is performed with a crossing combination between a male sterile line (mother plant) and a restorer line (father plant) selected in the present subject matter, and after a mature stage of about 30 days has passed, cactus fruits at a state of being not opened at the shells thereof are harvested. The cactus fruits having the shells are disinfected in a flame of ethanol of 70%.

Next, the pulps and seeds contained in the cactus fruits are selected and sterilized, and the sterilized seeds are sowed in petri dishes each having a diameter of 10 cm in a clean-bench, each petri dish having medium 30 ml divided thereinto. Then, the sowed seeds are cultured to obtain germinated seedlings, and the step of obtaining the seedlings is carried out in a constant temperature and humidity culture room of 26° C. on the conditions of intensity of illumination of 2,500 lux and daylength of 16 hours.

Next, only colored seedlings are selected from the germinated seedlings, and the selected colored seedlings are first grafted onto small virus-free Hylocereus trigonus in test tubes. After that, the Hylocereus trigonus onto which the seedlings are grafted are supportedly placed in media divided into sterilized test tubes, and the grafted seedlings are cultured in a culture room for 4 months. Next, light-shielding film is covered on the cultured seedlings to harden the seedlings, and the seedlings are secondarily grafted onto Hylocereus trigonus grown to a length of 14 cm to 15 cm in an outside greenhouse. In this step, the seedlings having a diameter in a range of 0.7 cm to 1 cm are selected to cultivate the new grafted cactus (Gymnocalycium mihanovichii) cultivar “Yellow Top” according to the present subject matter. According to the present subject matter, when the seedlings having such diameter range are grafted, the loss rate of the colored seedlings can be minimized, and further, most appropriate grafting time is obtained.

Hereinafter, an explanation on a method for cultivating a grafted cactus (Gymnocalycium mihanovichii) cultivar “Yellow Top” is in detail given with reference to the preferred embodiment of the present subject matter. However, it should be noted that the scope of the present subject matter is not limited to the examples. They should be considered as merely being illustrative and representative for the present subject matter.

### EXAMPLES

### Example 1

### Cultivation of a New Grafted Cactus (Gymnocalycium mihanovichii) Cultivar “Yellow Top”

Gymnocalycium mihanovichii “GG02451” having a yellow bulb and a green growing point was selected as a male sterile line (mother plant), and Gymnocalycium mihanovichii “GG205736” having an orange bulb was selected as a restorer line (father plant).

The combination is crossed, and after a mature stage of about 30 days had been passed, cactus fruits at a state of being not opened at the shells thereof were harvested. The cactus fruits having the shells were disinfected in a flame of ethanol.

Next, the seeds contained in the cactus fruits were selected and sterilized, and the sterilized seeds were sowed in petri dishes in a clean bench, each petri dish having medium 30 ml divided thereinto. Especially, the selected seeds were put and sterilized in the medium like Hyponex, Sucrose, Agar, and so on, and they were sowed by 50 seeds in the petri dishes in the clean bench, each petri dish having the medium of 30 ml divided thereinto and having a diameter of 10 cm. Then, the sowed seeds were cultured for 30 days in a culture room with constant temperature of 26° C. and humidity under the conditions of 2,500 lux of illumination intensity and 16 hours of day length, thereby obtaining 178 seedlings.

Next, only colored seedlings were selected from the germinated seedlings, and the selected colored seedlings were grafted onto small virus-free Hylocereus trigonus cultured in test tubes. After that, medium of 9 ml was divided in each of the sterilized test tubes, and the seedlings grafted onto the virus-free Hylocereus trigonus were transferred to sterilized test tubes. The grafted seedlings were cultured in the culture room having the above-mentioned conditions for 4 months.

Next, light-shielding film having light shielding capacity of 50% was covered on the cultured seedlings each having a diameter in a range of 0.7 cm to 1 cm to harden the seedlings for 2 weeks, and the seedlings were secondarily grafted onto Hylocereus trigonus having a length of 15 cm grown in an outside greenhouse, thereby selecting 128 grafted cactuses having high adaptability to environment and excellent ornamental values.

The grafted cactuses selected through the tests of the Cactus Research Institute of the present applicant were designated as a new cultivar through a general farm validation test and a cultivar exhibition by a new cultivar selection committee of Korea Rural Development Administration and they named it “Yellow Top”.

TABLE 1
Pedigree diagram of Gymnocalycium mihanovichii cultivar “Yellow Top”

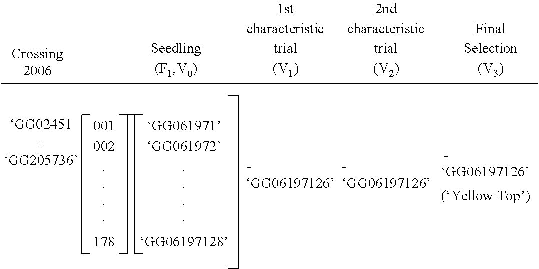

### Example 2

### Main Characteristics Between Control and New Cultivar “Yellow Top”

The grafted cactus (Gymnocalycium mihanovichii) cultivar “Yellow Top” cultivated by the method of the present subject matter had a round bulb shape and yellow color bulbs, which provides an excellent ornamental value. The grafted cactus of the present subject matter had yellow mother bulb and yellow cormlets and had RHS color chart No. 21A when compared with No. 15B of control. The grafted cactus of the present subject matter had 8 to 9 ribs and its spine was light brown and semi-erected. The spine length was 0.3 cm, which was the same as the control Gymnocalycium mihanovichii “Yellow King”. The grafted cactus of the present subject matter was able to produce 15 to 17 cormlets per plant, which had higher productivity than the control “Yellow King”.

TABLE 2
Morphological characteristics of Gymnocalycium mihanovichii cultivar “Yellow Top”.

| | | Bulb | No. of | Incision | | |
|---|---|---|---|---|---|---|
| Cultivar | Bulb color^{z} | shape | Ribs | shape | Spine color | Spine length |
| Yellow Top | yellow(21A) | round | 8~9 | triangle | light brown | 0.3 cm |
| Yellow King | yellow(15B) | round | 8~9 | round | brown | 0.3 cm |
| (control) | | | | | | |

^{z}RHS color chart

TABLE 3
Growth characteristics^{z} of grafted cactus Gymnocalycium mihanovichii
cultivar “Yellow Top”.

| | Bulb diameter increase(mm) | | | No. of offshoots | | |
|---|---|---|---|---|---|---|
| Cultivar | 1^{st} | 2^{nd} | 3^{rd} | 1^{st} | 2^{nd} | 3^{rd} |
| Yellow Top | 36.8 | 36.9 | 36.7 | 15.5 | 16.3 | 17.1 |
| Yellow King | 34.9 | 34.0 | 33.4 | 14.2 | 13.8 | 14.0 |
| (control) | | | | | | |

^{z}Each cultivar was grafted on a 15 cm length of Hylocereus trigonus and their Characteristics were investigated 6 months.

### Example 3

### Stability in Propagation Between Control and New Cultivar “Yellow Top”

As apparent from Table 4, the grafted cactus (Gymnocalycium mihanovichii) cultivar “Yellow Top” cultivated by the present method maintained their genetic traits even though years are increased, in the same manner as the control “Yellow King”. However, the grafted cactus cultivar “Yellow Top” had a light brown spine color, which was different from the control “Yellow King” having a brown spine color.

TABLE 4
Persistence of bulb color and bulb shape under the 3 years test.

| | | Bulb | Bulb | No. of | Incision | |
|---|---|---|---|---|---|---|
| Cultivar | Test year | color [symbol] . | shape | Ribs | shape | Spine color |
| Yellow | 1^{st}(2006) | yellow(21A) | round | 8~9 | triangle | light brown |
| Top | 2^{nd}(2007) | yellow(21A) | round | 8~9 | triangle | light brown |
| | 3^{rd}(2008) | yellow(21A) | round | 8~9 | triangle | light brown |
| Yellow | 1^{st}(2006) | yellow(15B) | round | 8~9 | round | brown |
| King | 2^{nd}(2007) | yellow(15B) | round | 8~9 | round | brown |
| (control) | 3^{rd}(2008) | yellow(15B) | round | 8~9 | round | brown |

[symbol]

. RHS Color chart

### Example 4

### Biological Characteristics Between Control and New Cultivar “Yellow Top”

As apparent from Table 5, according to the biological characteristics of the grafted cactus (Gymnocalycium mihanovichii) cultivar “Yellow Top” cultivated by the present method, the grafted cactus “Yellow Top” had a larger mother bulb size than the control and had a triangular incision shape different from that of the control “Yellow King”. Also, the grafted cactus “Yellow Top” had the number of cormlets larger than that of the control. Meanwhile, other biological characteristics of the grafted cactus cultivar “Yellow Top” were expressed in a similar manner to the control.

TABLE 5
Comparison of biological characteristics between
Gymnocalycium mihanovichii cultivar “Yellow Top” and control

| | | | | | | | | | | | | Yellow | | Yellow | |
|---|---|---|---|---|---|---|---|---|---|---|---|---|---|---|---|
| | | | | | | | | | | | | Top | | King | |
| | | | | | | | | | | | | | Experi- | | Experi- |
| | Character- | Expression | | | | | | | | | | | mental | | mental |
| No | istics | 1 | 2 | 3 | 4 | 5 | 6 | 7 | 8 | 9 | 10 | No | value | No | value |
| 1 | Mother | flat | round | flat- | oval | conical | crest | other | | | | 2 | — | 2 | — |
| (*) | bulb: | | | round | | | | | | | | | | | |
| (+) | shape | | | | | | | | | | | | | | |
| 2 | Mother | | | small | | Medium | | large | | | | 7 | 36.8 | 5 | 34.1 |
| (*) | bulb: | | | | | | | | | | | | | | |
| | size(mm) | | | | | | | | | | | | | | |
| 3 | Mother | single- | Bi- | | | | | | | | | 1 | — | 1 | — |
| (*) | bulb: | color | color | | | | | | | | | | | | |
| | color | | | | | | | | | | | | | | |
| | number | | | | | | | | | | | | | | |
| 4 | Mother | red | pink | orange | yellow | green | pur- | black | other | | | 4 | 21A | 4 | 15B |
| (*) | bulb: | | | | | | ple | | | | | | | | |
| | Color | | | | | | | | | | | | | | |
| 5 | Mother | | | | | | | | | | | — | — | — | — |
| | bulb: | | | | | | | | | | | | | | |
| | main | | | | | | | | | | | | | | |
| | color | | | | | | | | | | | | | | |
| | (base | | | | | | | | | | | | | | |
| | color in | | | | | | | | | | | | | | |
| | case of | | | | | | | | | | | | | | |
| | bi-color) | | | | | | | | | | | | | | |
| 6 | bi-color | red | pink | yellow | green | black | other | | | | | — | — | — | — |
| (*) | cultivar: | | | | | | | | | | | | | | |
| | mother | | | | | | | | | | | | | | |
| | bulb: | | | | | | | | | | | | | | |
| | pattern | | | | | | | | | | | | | | |
| | color | | | | | | | | | | | | | | |
| 7 | bi-color | hori- | spot | external | ver- | | | | | | | — | — | — | — |
| (*) | cultivar: | zontal | | | tical | | | | | | | | | | |
| (+) | mother | | | | | | | | | | | | | | |
| | bulb: | | | | | | | | | | | | | | |
| | pattern of | | | | | | | | | | | | | | |
| | bi-color | | | | | | | | | | | | | | |
| 8 | bi-color | No | | | | | | | | Yes | | — | — | — | — |
| (*) | cultivar: | | | | | | | | | | | | | | |
| | mother | | | | | | | | | | | | | | |
| | bulb: | | | | | | | | | | | | | | |
| | expression | | | | | | | | | | | | | | |
| | of second | | | | | | | | | | | | | | |
| | color of | | | | | | | | | | | | | | |
| | tip | | | | | | | | | | | | | | |
| 9 | bi-color | yellow | green | black | other | | | | | | | — | — | — | — |
| | cultivar: | | | | | | | | | | | | | | |
| | mother | | | | | | | | | | | | | | |
| | bulb: | | | | | | | | | | | | | | |
| | second | | | | | | | | | | | | | | |
| | color of | | | | | | | | | | | | | | |
| | tip (if any) | | | | | | | | | | | | | | |
| 10 | Mother | un- | | medium | | clear | | | | | | 1 | — | 1 | — |
| (*) | bulb: | clear | | | | | | | | | | | | | |
| | horizontal | | | | | | | | | | | | | | |
| | pattern | | | | | | | | | | | | | | |
| | coloring | | | | | | | | | | | | | | |
| 11 | rib: | very | | a | | medium | | many | | so | | 7 | 8.4 | 7 | 8.6 |
| | number | few | | few | | | | | | many | | | | | |
| 12 | Rib: | straight | curve | straight | curved | other | | | | | | 1 | — | 1 | — |
| (*) | shape | | | pro- | pro- | | | | | | | | | | |
| (+) | | | | truding | truding | | | | | | | | | | |
| 13 | incision: | | | shallow | | medium | | deep | | | | 7 | — | 7 | — |
| (*) | depth | | | | | | | | | | | | | | |
| 14 | incision: | triangluar | round | pro- | other | | | | | | | 1 | — | 2 | — |
| (*) | shape | | | truding | | | | | | | | | | | |
| (+) | | | | | | | | | | | | | | | |
| 15 | cormlet: | none or | | a few | | medium | | Many | | so | | 7 | 16.3 | 5 | 14.0 |
| (*) | number | very few | | | | | | | | many | | | | | |
| 16 | cormlet: | com- | general | pro- | | | | | | | | 3 | — | 3 | — |
| (+) | attached | pressed | | truding | | | | | | | | | | | |
| | shape | | | | | | | | | | | | | | |
| 17 | cormlet: | under | center | entire | irreg- | | | | | | | 2 | — | 2 | — |
| (*) | attached | center of | Of | mother | ular | | | | | | | | | | |
| (+) | position | mother | mother | bulb | | | | | | | | | | | |
| | | bulb | bulb | | | | | | | | | | | | |
| 18 | cormlet: | Single- | bi-color | | | | | | | | | 1 | — | 1 | — |
| (*) | color | color | | | | | | | | | | | | | |
| | number | | | | | | | | | | | | | | |
| 19 | cormlet: | Red | pink | orange | yellow | green | pur- | black | other | | | 4 | 21A | 4 | 15B |
| (*) | color | | | | | | ple | | | | | | | | |
| | (base | | | | | | | | | | | | | | |
| | color in | | | | | | | | | | | | | | |
| | case of | | | | | | | | | | | | | | |
| | bi-color) | | | | | | | | | | | | | | |
| 20 | cormlet: | | | | | | | | | | | — | — | — | — |
| | main color | | | | | | | | | | | | | | |
| | (base color | | | | | | | | | | | | | | |
| | in case of | | | | | | | | | | | | | | |
| | bi-color) | | | | | | | | | | | | | | |
| 21 | bi-color | red | pink | yellow | green | black | other | | | | | — | — | — | — |
| (*) | cultivar: | | | | | | | | | | | | | | |
| | cormlet: | | | | | | | | | | | | | | |
| | pattern | | | | | | | | | | | | | | |
| | color | | | | | | | | | | | | | | |
| 22 | spine: | no | | | | | | | | yes | | 9 | — | 9 | — |
| (*) | existence | | | | | | | | | | | | | | |
| 23 | spine: | | | short | | medium | | long | | | | 5 | 0.3 cm | 5 | 0.3 cm |
| | length (if | | | | | | | | | | | | | | |
| | exist) | | | | | | | | | | | | | | |
| 24 | spine: | erect | Semi- | ex- | twisted | | | | | | | 2 | — | 2 | — |
| (*) | shape | | erect | tended | | | | | | | | | | | |
| (+) | (if exist) | | | | | | | | | | | | | | |
| 25 | spine: | white | brown | black | other | | | | | | | 2 | — | 2 | — |
| | color | | | | | | | | | | | | | | |
| | (if color | | | | | | | | | | | | | | |
| | spine | | | | | | | | | | | | | | |
| | exist) | | | | | | | | | | | | | | |
| 26 | spine: | | | a few | | medium | | many | | | | 3 | — | 3 | — |
| (*) | areole | | | | | | | | | | | | | | |
| | trichome | | | | | | | | | | | | | | |

*RHS color chart

As set forth in the foregoing, the grafted cactus (Gymnocalycium mihanovichii) cultivar “Yellow Top” cultivated by the present method has a round and yellow bulb, showing excellent ornamental value, and they have yellow mother bulb and yellow cormlets, the yellow having RHS color chart No. 21A as compared with No. 15B of control. Further, the provided cultivar has 8 to 9 ribs and light brown and semi-erected spines, each spine having a length of 0.3 cm, which is the same as the control “Yellow King”, and produces 15 to 17 cormlets per plant, which has higher productivity than the control “Yellow King”.

Accordingly, it is found that the present subject matter can provide a method for cultivating the plants contained to new grafted cactus cultivars having yellow bulb.

In addition, through the present method, the cactuses having excellent ornamental values are cultivated and sold in the market, thereby advantageously increasing the productivity and gains of the cactus cultivating farms.

Although the present subject matter has been described with preferred embodiments, it is to be understood that variations and modifications may be resorted to as will be apparent to those skilled in the art. Such variations and modifications are to be considered within the purview and the scope of the claims appended hereto.

What is claimed is:

1. A method of cultivating a grafted cactus (Gymnocalycium mihanovichii) cultivar “Yellow Top” comprising:
a) selecting a cross combination between Gymnocalycium mihanovichii “GG02451” as a male sterile line (mother plant) having a yellow bulb and a green growing point, and Gymnocalycium mihanovichii “GG205736” as a restorer line (father plant) having an orange bulb;
b) crossing the combination, harvesting cactus fruits at a state of being not opened at the shells thereof after a mature stage has been passed, disinfecting the cactus fruits having the shells in a flame of ethanol, selecting and sterilizing seeds contained in the cactus fruits, and sowing the seeds in petri dishes in a clean-bench, each petri dish having medium divided thereinto;
c) culturing the sowed seeds to obtain germinated seedlings;
d) selecting only colored seedlings from the germinated seedlings;
e) first-grafting the selected colored seedlings onto small virus-free Hylocereus trigonus in test tubes, supportedly placing the Hylocereus trigonus onto which the seedlings are grafted in media divided into sterilized test tubes, and culturing the grafted seedlings in a culture room; and
f) covering light-shielding film on the cultured seedlings to harden the seedlings, secondarily grafting only the seedlings onto Hylocereus trigonus grown in an outside greenhouse, and cultivating the grafted seedlings in the outside greenhouse.

2. The method according to claim 1, wherein the obtaining of the seedlings in (c) is carried out in a constant temperature and humidity culture room of 26° C. and under the conditions of 2,500 lux of illumination intensity and 16 hours of day length.

3. The method according to claim 1, wherein the secondarily grafting of the seedlings in (f) comprises selecting the seedlings each having a diameter of about 1 cm.

4. A grafted cactus (Gymnocalycium mihanovichii) cultivar “Yellow Top” cultivated by the method according to claim 1.
